(19) United States
(12) Patent Application Publication
(10) Pub. No.: US 2023/0286979 A1
(43) Pub. Date: Sep. 14, 2023

(54) SALT OF DIHYDROPYRIDO[2,3-D]PYRIMIDINONE DERIVATIVE, PREPARATION METHOD THEREFOR, AND USE THEREOF

(71) Applicant: NANJING CHIA TAI TIANQING PHARMACEUTICAL CO., LTD., Nanjing (CN)

(72) Inventors: Changyou MA, Nanjing (CN); He TIAN, Nanjing (CN); Jianliang ZHAO, Nanjing (CN); Donghui CHEN, Nanjing (CN); Jian WU, Nanjing (CN); Dan XU, Nanjing (CN); Chunxia ZHU, Nanjing (CN); Zhoushan TIAN, Nanjing (CN)

(73) Assignee: NANJING CHIA TAI TIANQING PHARMACEUTICAL CO., LTD., Nanjing (CN)

(21) Appl. No.: 18/017,422

(22) PCT Filed: Jul. 22, 2021
(86) PCT No.: PCT/CN2021/107815

§ 371 Date: Jan. 23, 2023

(30) Foreign Application Priority Data
Jul. 22, 2020 (CN) 202010711260.5

(51) Int. Cl. C07D 471/04; A61P 35/00

(52) U.S. Cl. CPC C07D 471/04; A61P 35/00

(57) ABSTRACT

A salt of a dihydropyrido[2,3-d]pyrimidinone derivative, a preparation method thereof and the use thereof are provided. The salt is selected from fumarate, methanesulfonate, isethionate, α-naphthalenesulfonate, p-toluenesulfonate, 1,2-ethanedisulfonate, oxalate, maleate, citrate, succinate, L-(+)-tartrate, hippurate, L-ascorbate, L-malate, benzoate, gentisate, a hydrochloride, a sulfate or a phosphate. The salt of the dihydropyrido[2,3-d]pyrimidinone derivative of the present application can be used in the treatment of breast cancer, prostate cancer, or ovarian cancer.

[image]

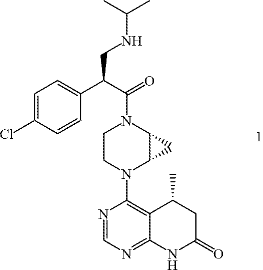 [SMILES: CC(C)NC[C@@H](C(=O)N1CCN(c2ncnc3c2[C@H](C)CC(=O)N3)[C@H]2C[C@H]21)c1ccc(Cl)cc1]

### CROSS REFERENCE TO THE RELATED APPLICATIONS

This application is the national phase entry of International Application No. PCT/CN2021/107815, filed on Jul. 22, 2021, which is based upon and claims priority to Chinese Patent Application No. 202010711260.5, filed on Jul. 22, 2020, the entire contents of which are incorporated herein by reference.

### TECHNICAL FIELD

The present application belongs to the field of medicinal chemistry, and specifically relates to a salt of a dihydropyrido[2,3-d]pyrimidinone derivative, a preparation method and medical use thereof.

### BACKGROUND

The PI3K/AKT/mTOR pathway consisting of phosphoinositide-3-kinase (PI3K) and its downstream protein AKT (also known as protein kinase B, PKB), and mammalian target of Rapamycin (mTOR) as a very important intracellular signal transduction pathway, the pathway exerts an extremely important biological function in the process of cell growth, survival, proliferation, apoptosis, angiogenesis, autophagy, etc. Abnormal activation of the pathway will cause a series of diseases such as cancer, neuropathy, autoimmune disease, and hemolymphatic system disease.

AKT is a type of serine/threonine kinase and affects the survival, growth, metabolism, proliferation, migration, and differentiation of cell through numerous downstream effectors. Overactivation of AKT has been observed in more than 50% of human tumors, especially in prostate cancer, pancreatic cancer, bladder cancer, ovarian cancer, and breast cancer. Overactivation of AKT may lead to the formation, metastasis, and drug resistance of tumor.

AKT has three isoforms: AKT1, AKT2, and AKT3. As a typical protein kinase, each isoform consists of an amino-terminal pleckstrin homology (PH) domain, a middle ATP-binding kinase domain, and a carboxyl-terminal regulatory domain. About 80% amino acid sequences of the three isoforms are homologous, and only the amino acid sequences in a binding domain between the PH domain and the kinase domain changes greatly.

The current drugs targeting the PI3K/AKT/mTOR signaling pathway mainly include PI3K inhibitors and mTOR inhibitors, while AKT is at the core of the signal transduction pathway. Inhibition of the AKT activity can not only avoid the severe side effects caused by inhibition of upstream PI3K, but also avoid the negative feedback mechanism caused by inhibition of downstream mTOR from affecting the efficacy of a drug. For example, CN101631778A discloses a class of cyclopenta[D]pyrimidine derivatives, CN101578273A discloses a class of hydroxylated and methoxylated cyclopenta[D]pyrimidine derivatives, CN101511842A discloses a class of dihydrofuropyrimidine derivatives, CN101970415A discloses a class of 5H-cyclopenta[d]pyrimidine derivatives, and these compounds inhibit AKT1 with IC₅₀ less than 10 µM. However, development of effective and selective AKT inhibitors is still an important direction for current development of tumor-targeting drugs.

### SUMMARY

In one aspect, the present application provides a pharmaceutically acceptable salt of a compound represented by formula 1, which is selected from a salt of organic acid or a salt of inorganic acid. The salt of organic acid is selected from a fumarate, a mesylate, an isethionate, an α-naphthalenesulfonate, a p-toluenesulfonate, a 1,2-ethanedisulphonate, an oxalate, a maleate, a citrate, a succinate, an L-(+)-tartrate, a hippurate, an L-ascorbate, an L-malate, a benzoate, or a gentisate, and the salt of inorganic acid is selected from a hydrochloride, a sulfate, and a phosphate, and the compound represented by formula 1 has the following structure:

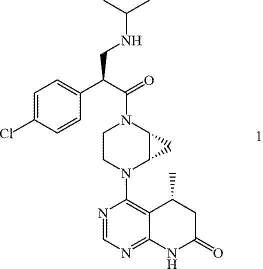 [SMILES: CC(C)NC[C@@H](C(=O)N1CCN(c2ncnc3c2[C@H](C)CC(=O)N3)[C@H]2C[C@H]21)c1ccc(Cl)cc1]

In some embodiments, the salt of organic acid is a fumarate.

In some embodiments, the salt of inorganic acid is a hydrochloride.

In some embodiments, in the salt of organic acid, a molar ratio of the compound represented by formula 1 to organic acid is 1: 1.

In some embodiments, in the hydrochloride, a molar ratio of the compound represented by formula 1 to hydrogen chloride is 1: 1 or 1: 2.

In some embodiments, in the hydrochloride, a molar ratio of the compound represented by formula 1 to hydrogen chloride is 1: 2.

In some embodiments, in the sulfate, a molar ratio of the compound represented by formula 1 to sulfuric acid is 1: 1.

In some embodiments, in the phosphate, a molar ratio of the compound represented by formula 1 to phosphoric acid is 1: 1.

It can be understood that the salt of the present application results from a salification reaction of the compound represented by formula 1 with a corresponding acid. In the reaction, the compound represented by formula 1 is converted into cations that bind to acid radicals of the corresponding acid to form the salt. Therefore, in the present application, a molar ratio of the compound represented by formula 1 to an acid can be understood as a molar ratio of cations of the compound represented by formula 1 to acid radicals of the corresponding acid.

In some typical embodiments, the present application provides a fumarate of the compound represented by formula 1, and a molar ratio of the compound represented by formula 1 to fumaric acid is 1: 1, or a molar ratio of cations of the compound represented by formula 1 to acid radicals of fumaric acid is 1: 1.

In some typical embodiments, the present application provides a hydrochloride of the compound represented by formula 1, a molar ratio of the compound represented by formula 1 to hydrogen chloride is 1: 1, or a molar ratio of cations of the compound represented by formula 1 to chloridion is 1: 1, and in this case, the hydrochloride is a monohydrochloride of the compound represented by formula 1.

In some typical embodiments, the present application provides a hydrochloride of the compound represented by formula 1, a molar ratio of the compound represented by formula 1 to hydrogen chloride is 1: 2, or a molar ratio of cations of the compound represented by formula 1 to chloride ions is 1: 2, and in this case, the hydrochloride is a dihydrochloride of the compound represented by formula 1.

In another aspect, the present application provides a preparation method of the pharmaceutically acceptable salt of the compound represented by formula 1, which comprising a step of salification reation of the compound represented by formula 1 with the corresponding acid.

In some embodiments, a solvent for salification reaction is selected from a mixed solvent of an alcohol solvent and an alkane solvent, a mixed solvent of a ketone solvent and an alkane solvent, a mixed solvent of an ester solvent and an alkane solvent, a mixed solvent of a benzene solvent and an alkane solvent, and a mixed solvent of a halogenated hydrocarbon solvent and an alkane solvent.

In some embodiments, the alcohol solvent is selected from methanol, ethanol or isopropanol, and preferably isopropanol; the ketone solvent is selected from acetone or butanone, and preferably acetone; the ester solvent is selected from ethyl acetate or butyl acetate, and preferably ethyl acetate; the benzene solvent is toluene; the halogenated hydrocarbon solvent is dichloromethane; and the alkane solvent is n-heptane.

In some typical embodiments, the present application provides a preparation method of a fumarate of the compound represented by formula 1, which comprising a step of salification reation of the compound represented by formula 1 with fumaric acid, and preferably, the solvent for the salificationreaction is a mixed solvent of isopropanol and n-heptane.

In some typical embodiments, the present application provides a preparation method of a hydrochloride of the compound represented by formula 1, which comprising a step of salification reation of the compound represented by formula 1 with hydrochloric acid, and preferably, the solvent for the salification reaction is selected from a mixed solvent of toluene and n-heptane and a mixed solution of ethyl acetate and n-heptane.

In another aspect, the present application also provides a pharmaceutical composition comprising the pharmaceutically acceptable salt of the compound represented by formula 1.

In some embodiments, the pharmaceutical composition further comprises one or more pharmaceutically acceptable carriers.

In some embodiments, the pharmaceutical composition is a solid preparation suitable for oral administration, and preferably tablets or capsules.

In another aspect, the present application also provides the pharmaceutically acceptable salt of the compound represented by formula 1 or a pharmaceutical composition thereof that is used as a medicament.

In another aspect, the present application also provides use of the pharmaceutically acceptable salt of the compound represented by formula 1 or a pharmaceutical composition thereof in the preparation of a medicament for preventing and/or treating an AKT protein kinase-mediated disease or disease state.

In another aspect, the present application also provides use of the pharmaceutically acceptable salt of the compound represented by formula 1 or a pharmaceutical composition thereof in the prevention and/or treatment of an AKT protein kinase-mediated disease or disease state.

In another aspect, the present application also provides a method for preventing and/or treating an AKT protein kinase-mediated disease or disease state, which comprising a step of administering the pharmaceutically acceptable salt of the compound represented by formula 1 or a pharmaceutical composition thereof of the present application to the subject in need.

In another aspect, the present application also provides the pharmaceutically acceptable salt of the compound represented by formula 1 or a pharmaceutical composition thereof of the present application that is used for preventing and/or treating an AKT protein kinase-mediated disease or disease state.

In some embodiments, the AKT protein kinase-mediated disease or disease state is cancer.

In some typical embodiments, the cancer is breast cancer, prostate cancer or ovarian cancer.

In some typical embodiments, the cancer is prostate cancer.

### Relevant Definitions

Unless otherwise specified, the following terms used in the description and claims have the following meanings.

The pharmaceutically acceptable salts of the present application also include their hydrate forms. The term “pharmaceutically acceptable carrier” refers to a carrier that has no obvious stimulating effect on the body and will not impair the biological activity and performance of an active compound. Pharmaceutically acceptable carriers include, but are not limited to, any diluent, disintegrant, adhesive, glidant, and wetting agent that have been approved by the National Medical Products Administration for human or animal use.

The term “fumaric acid” refers to trans-butenedioic acid having the following structure:

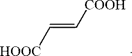

The term “alcohol solvent” refers to a derived substance resulting from the substitution of one or more hydrogen atoms on C1-C6 alkane with one or more hydroxyl groups (OH), and the C1-C6 alkane refers to straight-chain or branched-chain alkane containing 1-6 carbon atoms. Specific examples of alcohol solvents include, but are not limited to, methanol, ethanol, isopropanol or n-propanol.

The term “alkane solvent” refers to straight chain or branched or annular alkane containing 5-7 carbon atoms. Specific examples of alkane solvents include, but are not limited to, n-hexane, cyclohexane, n-heptane.

The term “ester solvent” refers to a chain compound containing the ester group -COOR and 3-10 carbon atoms, wherein R is C1-C6 alkyl, and the C1-C6 alkyl refers to straight-chain or branched-chain alkane containing 1-6 carbon atoms. Specific examples of ester solvents include, but are not limited to, methyl acetate, ethyl acetate, and propyl acetate.

The term “halogenated hydrocarbon solvent” refers to a derived substance resulting from the substitution of one or more hydrogen atoms on C1-C6 alkane with one or more halogen atoms, the C1-C6 alkane refers to straight-chain or branched-chain alkane containing 1-6 carbon atoms, and the halogen atom refers to fluorine, chlorine, bromine, iodine. Specific examples of halogenated hydrocarbon solvents include, but are not limited to, dichloromethane and chloroform.

The term “ketone solvent” refers to a chain or ring compound containing the carbonyl group -CO- and 3-10 carbon atoms. Specific examples of ketone solvents include, but are not limited to, acetone, butanone, and cyclohexanone.

The term “benzene solvent” refers to a solvent containing phenyl groups. Specific examples of benzene solvents include, but are not limited to, toluene, xylene, cumene or chlorobenzene.

The term “equivalent” refers to equivalent usage of other raw materials required in accordance with an equivalent relationship of a chemical reaction, taking a basic raw material used at each step as 1 equivalent.

Unless otherwise specified, the abbreviations in the present application have the following meanings:
M: mol/L
mM: mmol/L
nM: nmol/L
Boc: tert-butoxycarbonyl
DCM: dichloromethane
DEA: diethylamine
DIEA: N,N-diisopropylethylamine
HATU: 2-(7-azabenzotriazol)-N,N,N′,N′-tetramethyluronium hexafluorophosphate
RT: retention time
SFC: supercritical fluid chromatography
h: hour
min: minute
TK: tyrosine kinase
SEB: fluorescent signal enhancer
HTRF: homogeneous time resolved fluorescence
DTT: dithiothreitol

### BRIEF DESCRIPTION OF THE DRAWINGS

In order to more clearly describe the technical solutions of the examples of the present application and the prior art, the drawings that need to be used in the examples and the prior art will be briefly introduced below. Obviously, the drawings in the following description are some embodiments of the present application only, and those skilled in the art may also obtain other drawings according to these drawings.

FIG. 1 is a schematic diagram of a single molecule of a compound represented by formula 1 of Example 1;

FIG. 2 is a schematic diagram of asymmetric structural unit of an oxalate single crystal of the compound represented by formula 1 of Example 1;

FIG. 3 is an XRPD pattern of a sulfate of the compound represented by formula 1 of Example 2;

FIG. 4 is an XRPD pattern of a phosphate of the compound represented by formula 1 of Example 2;

FIG. 5 is an XRPD pattern of an isethionate of the compound represented by formula 1 of Example 2;

FIG. 6 is an XRPD pattern of an α-naphthalenesulfonate of the compound represented by formula 1 of Example 2;

FIG. 7 is an XRPD pattern of an L-malate of the compound represented by formula 1 of Example 2;

FIG. 8 is an XRPD pattern of a monohydrochloride of the compound represented by formula 1 of Example 3;

FIG. 9 is an XRPD pattern of a dihydrochloride of the compound presented by formula 1 of Example 4; and

FIG. 10 is an XRPD pattern of a fumarate of the compound represented by formula 1 of Example 5.

### DETAILED DESCRIPTION OF THE EMBODIMENTS

The present application will be described in more detail below with reference to embodiments. However, these specific descriptions are for the purpose of describing the technical solutions of the present application only, and are not intended to limit the present application in any manner.

### Example 1 Preparation of a Compound Represented by Formula 1

Preparation Example 1 Preparation of intermediate (R)-4-chloro-5-methyl-5,8-dihydropyrido[2,3-d]pyrimidin-7(6H)-one

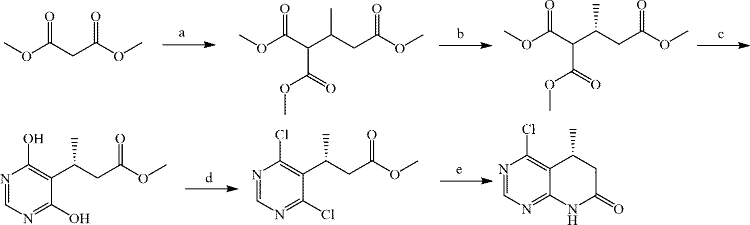

### A) Trimethyl 2-Methylpropane-1,1,3-Tricarboxylate

Under the protection of nitrogen gas, a sodium methylate-methanol solution (30 wt%, 50.32 g) was added to methanol (900 mL), the mixture was heated to 70° C., dimethyl malonate (461.12 g) and ethyl crotonate (349.46 g) were mixed until uniform and dropwise added to the above sodium methylate-methanol solution, and the reaction solution reacted at 70° C. for 3 h. After the reaction was completed, the reaction solution was evaporated under reduced pressure to remove the solvent, ethyl acetate (1 L) was added, the mixture was regulated with 4 M hydrochloric acid until the pH of the mixture was 7-8, water (500 mL) was added, and the solution was separated and evaporated under reduced pressure to remove the organic phase so as to yield a yellow liquid (777.68 g). ¹H NMR (400 MHz, DMSO-d₆) δ (ppm) 3.67 (s, 3H), 3.65 (s, 3H), 3.59 (s, 3H), 3.56 (d, J=6.8 Hz, 1H), 2.45-2.58 (m, 2H), 2.23-2.29 (m, 1H), 0.93 (d, J=6.8 Hz, 3H).

### B) Trimethyl (R)-2-Methylpropane-1,1,3-Tricarboxylate

Disodium hydrogen phosphate (4.5 g) was dissolved in deionized water (1.5 L) at 25° C., the solution was regulated with 2 N hydrochloric acid until the pH of the solution was 7.05, trimethyl 2-methylpropane-1,1,3-tricarboxylate (150.46 g) and lipase (Candida rugosa, 40 g, added in 6 d) were added, the mixture was regulated with a 2 N sodium hydroxide solution until the pH of the mixture was 7.0-7.6, and the reaction solution reacted at 35° C. for 6 d. Chirality detection ee%>98%, and chirality detection conditions: Chiralpak IC, 4.6×250 mm, 5 µm, and n-hexane: ethanol=9: 1 (volume ratio). The reaction solution was cooled to 10° C. and regulated with 3 M hydrochloric acid until the pH of the reaction solution was 3-4, ethyl acetate (500 mL) was added, the mixture was subjected to suction filtration, an obtained filter cake was washed with ethyl acetate (600 mL), the solution was separated, a saturated sodium bicarbonate aqueous solution (100 mL) was added for washing, the solution was separated, and an obtained organic phase was concentrated to yield a pale-yellow liquid (26.89 g). ¹H NMR (400 MHz, CDCl₃) δ (ppm) 3.74 (s, 6H), 3.68 (s, 3H), 3.46 (d, J=7.2 Hz, 1H), 2.71-2.79 (m, 1H), 2.54 (dd, J=15.6, 4.8 Hz, 1H), 2.32 (dd, J=16.0, 8.4 Hz, 1H), 1.06 (d, J=6.8 Hz, 3H).

### C) Methyl (R)-3-(4,6-Dihydroxypyrimidin-5-Yl)Butanoate

Under the protection of nitrogen gas, formamidine acetate (11.33 g) was dissolved in methanol (200 mL) at 20° C., the solution was cooled to 0° C., a sodium methylate-methanol solution (30 wt%, 55.62 g) was dropwise added, the reaction solution reacted at 0° C. for 60 min, a methanol (60 mL) solution of trimethyl (R)-2-methylpropane-1,1,3-tricarboxylate (24.07 g) was dropwise added, and the reaction solution was naturally heated to 20° C. and reacted for 10 h. After the reaction was completed, the reaction solution was cooled to 0° C., regulated with 3 N hydrochloric acid until the pH of the reaction solution was 5-6, evaporated under reduced pressure to remove the solvent, cooled to 0° C., and regulated with 3 N hydrochloric acid until the pH of the reaction solution was 3, after a solid was precipitated, the reaction solution was subjected to suction filtration to collect the solid, and an obtained filter cake was washed with ice water (100 mL) and dried in vacuum to yield a white solid (18.79 g) that was directly used at the next step.

### D) Methyl (R)-3-(4,6-Dichloropyrimidin-5-Yl)Butanoate

Under the protection of nitrogen gas, methyl (R)-3-(4,6-dihydroxypyrimidin-5-yl)butanoate (14.63 g) was dispersed into acetonitrile (70 mL) at 22° C., phosphorus oxychloride (26.42 g) and diisopropylethylamine (12.51 g) were dropwise added in sequence, the system released heat obviously and was heated to 60° C., the solids were gradually fully dissolved, and the reaction solution reacted for 18 h. After the reaction was completed, the reaction solution was cooled to 0° C., ethyl acetate (100 mL) was added, the mixture was regulated with a saturated sodium bicarbonate solution until the pH of the mixture was 7-8, extracted with ethyl acetate (50 mL × 3), and evaporated under reduced pressure to remove the organic phase so as to yield a yellow solid (13.89 g) that was directly used at the next step.

### E) (R)Chloro-5-Methyl-5,8-Dihydropyrido[2,3-d]Pyrimidin-7(6H)-One

Methyl (R)-3-(4,6-dichloropyrimidin-5-yl)butanoate (13.89 g) and ammonia water (25-28 wt%, 70 mL) were placed in a 100 mL high-pressure kettle at 20° C., and the reaction solution was heated to 50° C. and reacted for 18 h. After the reaction was completed, the reaction solution was cooled to 0° C. and subjected to suction filtration, and an obtained filter cake was beaten with a mixture (30 mL) of petroleum ether and ethyl acetate in a volume ratio of 10: 1 to yield a pale-yellow solid (7.32 g). LC-MS (ESI) m/z: 198 (M+H). ¹H NMR (300 MHz, CDCl₃) δ (ppm) 1.30 (d, J=7.2 Hz, 3H), 2.65-2.69 (m, 1H), 2.86-2.92 (m, 1H), 3.47-3.54 (m, 1H), 8.64 (s, 1H), 10.10 (s, 1H).

Preparation Example 2 Preparation of (R)-4-((1S,6R)-5-((S)-2-(4-chlorophenyl)-3-(isopropylamino)propionyl)-2,5-diazabicyclo[4.1.0]heptan-2-yl)-5-methyl-5,8-dihydropyrido[2,3-d]pyrimidin-7(6H)-one (compound represented by formula 1)

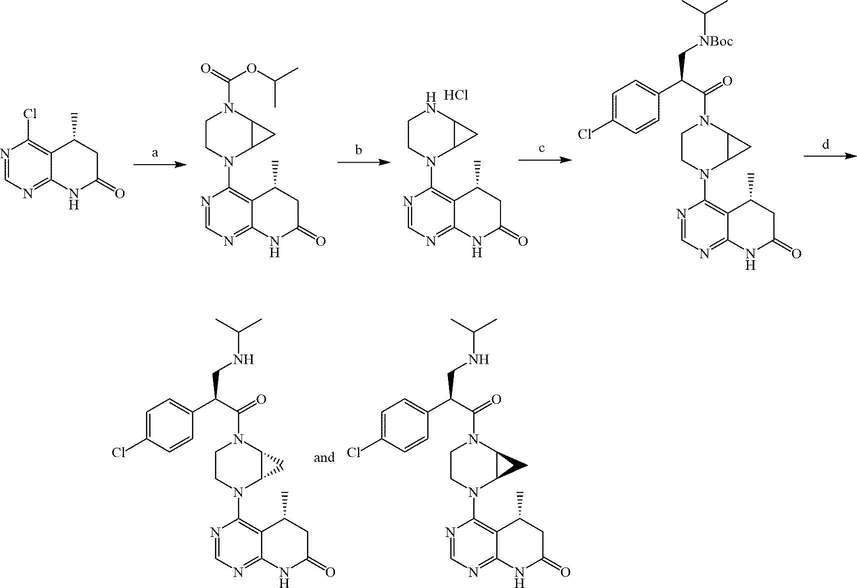

Reaction conditions: a) tert-butyl 2,5-diazabicyclo[4.1.0]heptane-2-carboxylate, N-methylpyrrolidone, and 4-dimethylaminopyridine; b) hydrogen chloride/1,4-dioxane (4.0 M) and dichloromethane; c) (S)-3-((tert-butoxycarbonyl)(isopropyl)amino)-2-(4-chlorophenyl)-propionic acid, 2-(7-benzotriazole oxide)-N,N,N′,N′-tetramethyluronium hexafluorophosphate, 4-dimethylaminopyridine, and N,N-dimethylformamide; and d) trifluoroacetic acid and dichloromethane.

### A) Tert-Butyl 5-((R)-5-Methyl-7-Oxo-5,6,7,8-Tetrahydropyrido[2,3-d]Pyrimidin-4-Yl)-2,5-Diazabicyclo[4.1.0]Heptane-2-Carboxylate

Under the protection of nitrogen gas, (R)-4-chloro-5-methyl-5,8-dihydropyrido[2,3-d]pyrimidin-7(6H)-one (0.21 g), tert-butyl 2,5-diazabicyclo[4.1.0]heptane-2-carboxylate (0.31 g), and 4-dimethylaminopyridine (0.39 g) were dissolved in N-methylpyrrolidone (5 mL) at 22° C., and the reaction solution was heated to 140° C. and reacted for 3 h. After the reaction was completed, the reaction solution was cooled to 20° C., poured into ice water (20 mL), extracted with ethyl acetate (20 mL × 2), washed with a saturated salt solution (10 mL × 3), evaporated under reduced pressure to remove the solvent, and separated by silica gel column chromatography (petroleum ether: ethyl acetate=(3: 1)-(1: 1)) to yield a pale-yellow liquid (0.28 g). LC-MS (ESI) m/z: 360 (M+H).

### B) (5R)(2,5-Diazabicyclo[4.1.0]Heptan-2-Yl)-5-Methyl-5,8-Dihydropyrido[2,3-d]Pyrimidin-7(6H)-One Hydrochloride

Tert-butyl 5-((R)-5-methyl-7-oxo-5,6,7,8-tetrahydropyrido[2,3-d]pyrimidin-4-yl)-2,5-diazabicyclo[4.1.0]heptane-2-carboxylate (0.28 g) was dissolved in dichloromethane (5 mL) at 20° C., hydrogen chloride/1,4-dioxane (4.0 mL) was added, and the reaction solution reacted for 1 h. After the reaction was completed, the reaction solution was evaporated under reduced pressure to remove the solvent so as to yield a yellow solid (0.23 g) that was directly used at the next step.

### C) Tert-Butyl (2S)-2-(4-Chlorophenyl)-3-(5-((R)-5-Methyl-7-Oxo-5,6,7,8-Tetrahydropyrido[2,3-d]Pyrimidin-4-yl)-2,5-Diazabicyclo[4.1.0]Heptan-2-yl)-3-Oxopropyl)(Isopropyl)Carbamate

Under the protection of nitrogen gas, (5R)-4-(2,5-diazabicyclo[4.1.0]heptan-2-yl)-5-methyl-5,8-dihydropyridin[2,3-d]pyrimidin-7(6H)-one hydrochloride (0.20 g) and (S)-3-((tert-butoxycarbonyl)(isopropyl)amino)-2-(4-chlorophenyl)-propionic acid (0.22 g) were dissolved in N,N-dimethylformamide (5 mL) at 20° C., 2-(7-benzotriazole oxide)-N,N,N′,N′-tetramethyluronium hexafluorophosphate (0.59 g) and 4-dimethylaminopyridine (0.48 g) were added, and the reaction solution reacted at 25° C. for 4 h. After the reaction was completed, water (20 mL) was added to the reaction solution, the mixture was extracted with ethyl acetate (10 mL × 3), an obtained organic phase was washed with a saturated salt solution (10 mL × 2), and the solution was evaporated under reduced pressure to remove the organic phase and separated by column chromatography (dichloromethane: methanol=50: 1) to yield a yellow solid (0.18 g). LC-MS (ESI) m/z: 583 (M+H).

### D) (R)((1S,6R)-5-((S)-2-(4-Chlorophenyl)-3-(Isopropylamino)Propionyl)-2,5-Diazabicyclo[4.1.0]Heptan-2-yl)-5-Methyl-5,8-Dihydropyrido[2,3-d]Pyrimidin-7(6H)-One

Tert-butyl (2S)-2-(4-chlorophenyl)-3-(5-((R)-5-methyl-7-oxo-5,6,7,8-tetrahydropyrido[2,3-d]pyrimidin-4-yl)-2,5-diazabicyclo[4.1.0]heptan-2-yl)-3-oxopropyl)(isopropyl)carbamate (0.18 g) was dissolved in dichloromethane (2 mL) at 20° C., trifluoroacetic acid (0.86 mL) was added, and the reaction solution reacted for 3 h. After the reaction was completed, dichloromethane (10 mL) was added to the reaction solution, a 2 M sodium hydroxide solution was dropwise added at 0° C. to regulate the pH of the mixture to 12, the solution was separated, an obtained organic phase was washed with a saturated salt solution (5 mL), and the solution was dried with anhydrous sodium sulfate and evaporated under reduced pressure to remove the organic phase so as to yield a yellow solid (0.10 g). The yellow solid was resolved by preparative high-performance liquid chromatography to yield isomer 1 (3 mg) and isomer 2 (12 mg). Preparative high-performance liquid chromatography conditions: chromatographic column: Aglient 5 µm prep-C18 50×21.2 mm; mobile phase A: water (containing 0.1 vol% of ammonium water (25-28 wt%)); and mobile phase B: methanol. Gradient: time: 0-10 min, 60-70% (volume percentage) of B phase.

Isomer 1: RT₁=5.3 min; LC-MS (ESI) m/z: 483 (M+H).

Isomer 2: RT=5.9 min; LC-MS (ESI) m/z: 483 (M+H); ¹H NMR (400 MHz, CDCl₃) δ (ppm) 8.27 (d, J=7.6 Hz, 1H), 7.92 (s, 1H), 7.27-7.30 (m, 4H), 4.23-4.29 (m, 1H), 3.90-3.95 (m, 1H), 3.81-3.85 (m, 1H), 3.69-3.72 (m, 1H), 3.44-3.59 (m, 1H), 3.20-3.38 (m, 3H), 3.01-3.05 (m, 1H), 2.70-2.85 (m, 3H), 2.47-2.57 (m, 1H), 2.21-2.25 (m, 1H), 1.25-1.28 (m, 3H), 1.03-1.11 (m, 6H), 0.82-0.90 (m, 2H).

In the present application, configurations of the compounds of Example 1 were determined by single crystal diffraction, and it was determined that isomer 2 was the compound represented by formula 1 of the present application:

Preparation of a single crystal: isomer 2 (30.0 mg) and isopropanol (2.0 mL) were placed in a 5 mL screw flask and stirred for 5 min until the solid was fully dissolved. Oxalic acid dihydrate (3.9 mg) was weighed and placed in the above flask, a white solid was gradually precipitated in the flask, the reaction solution was stirred at the room temperature for 3 h, and a large amount of white solid was precipitated in the flask. Methanol (1.0 mL) was placed in the flask, the white solid gradually disappeared, and after becoming clear, the solution was stirred for 1 h. The solution was filtered with a 0.22 µm microfiltration membrane to a 3 mL screw flask, and the opening of the flask was covered with a plastic wrap. The plastic warp covering the opening of the flask was pierced by a needle to form 8 small holes, the flask was placed at the room temperature for 7 d, and an oxalate single crystal of isomer 2 was obtained.

Single crystal diffraction experiment:
Single crystal X-ray diffractometer: BRUKER D8 VENTURE PHOTON II
Wavelength: Ga Kα (λ=1.34139 Å)
Test temperature: 190 K
Computer program for structural analysis: SHELXI,-2018

Single crystal data: molecular formula: C₅₅H₇₂C1₂N₁₂O₉; molecular weight: 1116.14; crystal system: hexagonal crystal system; space group: P61; unit cell parameters: a=25.8406(15) Å, b=25.8406(15) Å, c=45.916(3) Å, α=90°, β=90°, and γ=120°; unit cell volume: V=26552(4) Å³; the number of molecular formulas contained in the unit cell: Z=12; calculated density: D_{calc}=0.838 g/cm³; R(Fₒ): 0.0730; Rw(Fₒ²): 0.2069; goodness of fit (S): 1.034; and Flack parameter: 0.066(9).

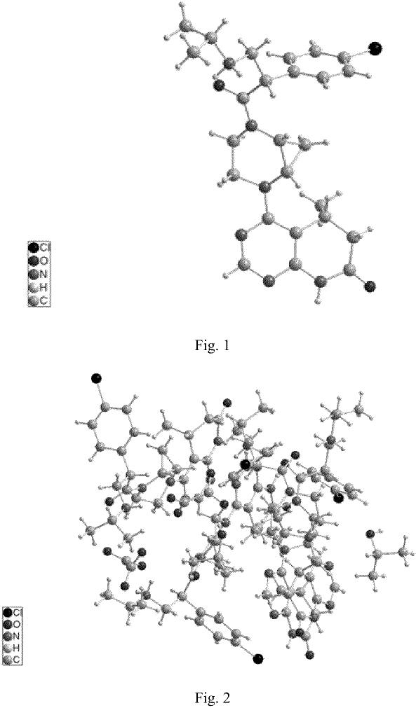

Structural description: single crystal X-ray diffraction and structural analysis show that the prepared single crystal is an oxalate isopropoxide of isomer 2. Asymmetric building blocks of the crystal include four isomer 2 molecules, two oxalic acid molecules, and two isopropanol molecules, and isomer 2 and oxalic acid form an oxalate. The single molecule of isomer 2 is shown in FIG. 1, and the asymmetric structural unit of the oxalate single crystal are shown in FIG. 2. The structural formula is shown below:

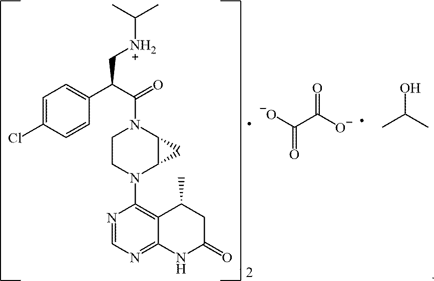

### Test Example 1 Test of AKT Kinase Inhibiting Activity

### 1. Materials and Reagents

Envision model plate reader (Molecular Devices)
White 384-well plate (Thermo, Art. No. #264706)
Main reagents included in an HTRF kinEASE TK kit (Cisbio, Art. No. #62TKOPEC)
TK-biotin substrate
Streptavidin-XL665
Europium-labeled tyrosine kinase substrate antibody
5× enzyme reaction buffer
SEB
HTRF assay buffer
AKT1 (Carna, Art. No. #01-101)
AKT2 (Carna, Art. No. #01-102)
AKT3 (Invitrogen, Art. No. #PV3185)
10 mM ATP (Invitrogen, Art. No. #PV3227)
1 M DTT (Sigma, Art. No. #D5545)
1 M MgCl₂ (Sigma, Art. No. #M8266)
Isomer 1 and isomer 2 of Example 1 of the present application
Positive control: GDC-0068

### 2. Experimental Procedure

### 2.1 Preparation of Reagents

TABLE 1

Concentrations of components of kinase reaction systems

| | | | | |
|---|---|---|---|---|
| Reaction reagent | | AKT1 | AKT2 | AKT3 |
| Concentration of enzyme | Final concentration at the enzyme reaction step (10 µL) | 0.6 ng/well | 0.1 ng/well | 0.3 ng/well |
| Concentration of ATP | | 2 µM | 20 µM | 10 nM |
| Concentration of TK-biotin substrate | | 2 µM | 2 µM | 2 µM |
| Enzyme reaction time | | 50 min | 50 min | 50 min |
| Concentration of streptavidin-XL665 | Final concentration in the overall reaction (20 µL) | 125 nM | 125 nM | 125 nM |
| Concentration of europium-labeled tyrosine kinase substrate antibody | | 1: 100 diluted | 1: 100 diluted | 1: 100 diluted |

### 1× Kinase Reaction Buffer

A 1× kinase reaction buffer for 1 mL of kinase AKT1, AKT2 or AKT3 included 200 µL of 5× kinase reaction buffer, 5 µL of 1 M MgC1₂, 1 µL of 1 M DTT, and 794 µL of ultra-pure water.

### 5× TK-Biotin Substrate and ATP Working Solution

Specific concentrations of the TK-biotin substrate and ATP are shown in Table 1.

The substrate and ATP were respectively diluted with the 1× kinase reaction buffer to a concentration 5 times the reaction concentration.

### 5× Kinase Working Solution

The concentration for enzyme screening is shown in Table 1. A 5× enzyme working solution was prepared from the 1× kinase reaction buffer.

### 4× Streptavidin-XL665 Working Solution

The concentration of streptavidin-XL665 in the reaction is shown in Table 1. A 4× streptavidin-XL665 working solution was prepared from the assay buffer.

4× europium-labeled tyrosine kinase substrate antibody working solution The europium-labeled tyrosine kinase substrate antibody was 100-fold diluted with the assay reaction buffer to obtain a working solution.

### 2.2 Experimental Process

After all the reagents were prepared according to the above method, except for the enzyme, the reagents were equilibrated to the room temperature and loaded.

a) first, a compound stock solution (10 mM DMSO solution) was diluted with DMSO to obtain a 100 µM compound solution, the compound solution was diluted with the 1× kinase reaction buffer to obtain a 2.5 µM compound working solution (containing 2.5% DMSO). A 2.5% DMSO solution was prepared from the 1× kinase reaction buffer, and the 2.5 µM compound working solution was diluted 7 times with the 2.5% DMSO solution according to a 4-fold gradient to obtain compound working solutions at 8 concentrations (2500 nM, 625 nM, 156 nM, 39 nM, 9.8 nM, 2.4 nM, 0.6 nM, and 0.15 nM). Except for control wells, 4 µL of diluted compound working solution was placed in each reaction well, and 4 µL of previously prepared 2.5% DMSO/kinase buffer was placed in each control well.

b) 2 µL of previously prepared TK-biotin substrate solution (the concentration of the substrate for enzyme screening is shown in Table 1) was placed in each reaction well.

c) 2 µL of previously prepared enzyme solution (the concentration of the enzyme is shown in Table 1) was placed in each reaction well except for negative wells, and 2 µL of 1× kinase reaction buffer corresponding to the enzyme was placed in each negative well to make up the volume. The plate was sealed with a sealing film, and the reaction solution was mixed until uniform and incubated at the room temperature for 10 min to allow the compound to fully react with and bind to the enzyme.

d) 2 µL of ATP solution was placed in each reaction well to initiate a kinase reaction (the concentration of ATP for enzyme screening and reaction time are shown in Table 1).

e) 5 min before the kinase reaction was completed, an assay solution was prepared. Streptavidin-XL665 and a europium-labeled tyrosine kinase substrate antibody (1: 100) assay solution (the concentration of the assay reagent is shown in Table 1) were prepared from the assay buffer in the kit.

f) After the kinase reaction was completed, 5 µL of diluted streptavidin-XL665 was placed in each reaction well and mixed with the reaction solution until uniform, and the diluted europium-labeled tyrosine kinase substrate antibody assay solution was immediately added.

g) The plate was sealed, the reaction solution was mixed until uniform and reacted at the room temperature for 1 h, and fluorescence signals were detected by using an ENVISION (Perkinelmer) instrument (320 nm stimulation, 665 nm, 615 nm emission). An inhibition rate in each well was calculated from all active wells and background signal wells, a mean value of repetitive wells was calculated, and the half inhibitory activity (IC50) of each compound to be tested was fitted by using the professional drawing analysis software PRISM 6.0.

TABLE 2

Experimental loading process

| | Kinase reaction system | Control group | |
|---|---|---|---|
| Enzyme reaction step (10 µL) | Sample group | Negative control | Positive control |
| Isomer 1 or isomer 2 | 4 µL | 4 µL of 2.5% DMSO/kinase buffer | 4 µL of 2.5% DMSO/kinase buffer |
| TK-biotin-labeled substrate | 2 µL | 2 µL | 2 µL |
| Kinase | 2 µL | 2 µL of kinase buffer | 2 µL |
| Seal with a film, and incubate at the room temperature for 10 min | | | |
| ATP | 2 µL | 2 µL | 2 µL |
| Seal with a film, and incubate at the room temperature for 50 min | | | |
| Detection steps (10 µL) | | | |
| Europium-labeled tyrosine kinase substrate antibody | 5 µL | 5 µL | 5 µL |
| Seal with a film, and incubate at the room temperature for 1 h | | | |
| Detection light: 320 nm, emitted light: 665 nm, 615 nm | | | |
| Streptavidin-XL665 | 5 µL | 5 µL | 5 µL |

### 2.3 Data Analysis

$$\text{ER = fluorescence value at 665 nm} / \text{fluorescence value at 615 nm}$$

$$\begin{matrix}\text{Inhibition rate =} \\ \left({\text{ER}}_{\text{positive control}} {\text{- ER}}_{\text{sample}}\right) / \left({\text{ER}}_{\text{positive control}} {\text{- ER}}_{\text{negative control}}\right) \times \\ 100 \%\end{matrix}$$

### 3. Experimental Results

Experimental results are shown in Table 3.

TABLE 3

AKT inhibiting activity

| Compound | Chemical structure | AKT1 enzyme activity IC₅₀ (nM) | AKT2 enzyme activity IC₅₀ (nM) | AKT3 enzyme activity IC₅₀ (nM) |
|---|---|---|---|---|
| Isomer 1 of Example 1 | 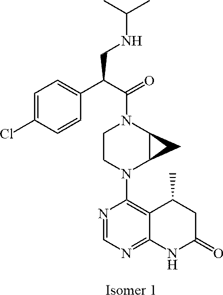 | 62 | 542 | 13 |
| Isomer 2 of Example 1 | 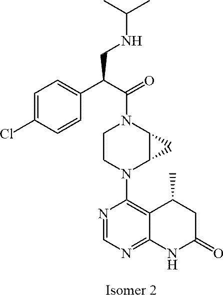 | 0.35 | 6.3 | 0.09 |
| Positive control GDC-0068 | 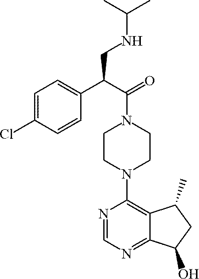 [SMILES: CC(C)NC[C@@H](C(=O)N1CCN(c2ncnc3c2[C@H](C)C[C@H]3O)CC1)c1ccc(Cl)cc1] | 3.2 | 1.7 | 2.5 |

### Example 2 Preparation of Salts of the Compound Represented by Formula 1

About 25 mg of compound represented by formula 1 and 1.05 equivalents of acid (for hydrochloric acid, a case of 2.10 equivalents was also set) were respectively added to 1 mL of solvent, and the reaction solution was stirred at the room temperature for 2 d. An obtained clear solution was attempted to crystallize by stirring at 5° C. and slow evaporation, and a solid was separated by centrifugation, blast-dried or dried under reduced pressure at 40° C. for 2-5 h, and characterized by XRPD and ¹HNMR. Salinization results are shown in the table below. Salt form was determined by XRPD, and molar ratio of free base of the compound represented by formula 1 to acid radicals (i.e., a molar ratio of cations of the compound represented by formula 1 to acid radicals) was determined by ¹HNMR.

TABLE 4

Salinization results of the compound represented by formula 1

| Acid | Solvent for a salt forming reaction^{∗} | Result | |
|---|---|---|---|
| | | Salt form | Salinization results (molar ratio of free alkali of the compound represented by formula 1 to acid radicals) |
| Sulfuric acid | Dichloromethane/n-heptane | Amorphous | Sulfate (1: 1) |
| Hydrobromic acid | Isopropanol/n-heptane; or acetone/n-heptane; or ethyl acetate/n-heptane; or toluene/n-heptane; or dichloromethane/n-heptane | | No salt is formed |
| Phosphoric acid | Isopropanol/n-heptane | Amorphous | Phosphate (1: 1) |
| Methanesulfonic acid | Ethyl acetate/n-heptane | Amorphous | Mesylate (1: 1) |
| Acetic acid | Isopropanol/n-heptane; or acetone/n-heptane; or ethyl acetate/n-heptane; or toluene/n-heptane; or dichloromethane/n-heptane | | No salt is formed |
| Isethionic acid | Ethyl acetate/n-heptane | Amorphous | Isethionate (1: 1) |
| α-naphthalenesulfonic acid | Ethyl acetate/n-heptane | Amorphous | α-naphthalenesulfonate (1: 1) |
| p-toluenesulfonic acid | Toluene/n-heptane | Amorphous | p-toluenesulfonate (1: 1) |
| 1,2-ethanedisulfonic acid | Isopropanol/n-heptane | Amorphous | 1,2-ethanedisulfonate (1: 1) |
| Oxalic acid | Toluene/n-heptane | Amorphous | Oxalate (1: 1) |
| Maleic acid | Isopropanol/n-heptane | Amorphous | Maleate (1: 1) |
| Fumaric acid | Isopropanol/n-heptane | Amorphous | Fumarate (1: 1) |
| Citric acid | Acetone/n-heptane | Amorphous | Citrate (1: 1) |
| Succinic acid | Isopropanol/n-heptane | Amorphous | Succinate |
| L-glutamic acid | Toluene/n-heptane | Amorphous | L-glutamate (1: 1) |
| L-tartaric acid | Isopropanol/n-heptane | Amorphous | L-tartrate (1: 1) |
| D-glucuronic acid | Toluene/n-heptane | Amorphous | D-glucuronate (1: 1) |
| Hippuric acid | Ethyl acetate/n-heptane | Mixture of amorphous and crystal | Hippurate (1: 1) |
| L-ascorbic acid | Acetone/n-heptane | Mixture of amorphous and crystal | L-ascorbate (1: 1) |
| L-malic acid | Acetone/n-heptane | Amorphous | L-malate (1: 1) |
| Benzoic acid | Ethyl acetate/n-heptane | Amorphous | Benzoate (1: 1) |
| Gentisic acid | Isopropanol/n-heptane | Mixture of amorphous and crystal | Gentisate (1: 1) |
| Hydrochloric acid | Toluene/n-heptane | Amorphous | Hydrochloride (1: 2) |
| Hydrochloric acid | Ethyl acetate/n-heptane | Amorphous | Hydrochloride (1: 1) |

^{∗}: in the solvents for salification reaction, a volume ratio of isopropanol, acetone, ethyl acetate, toluene and dichloromethane to n-heptane is 1: 2.

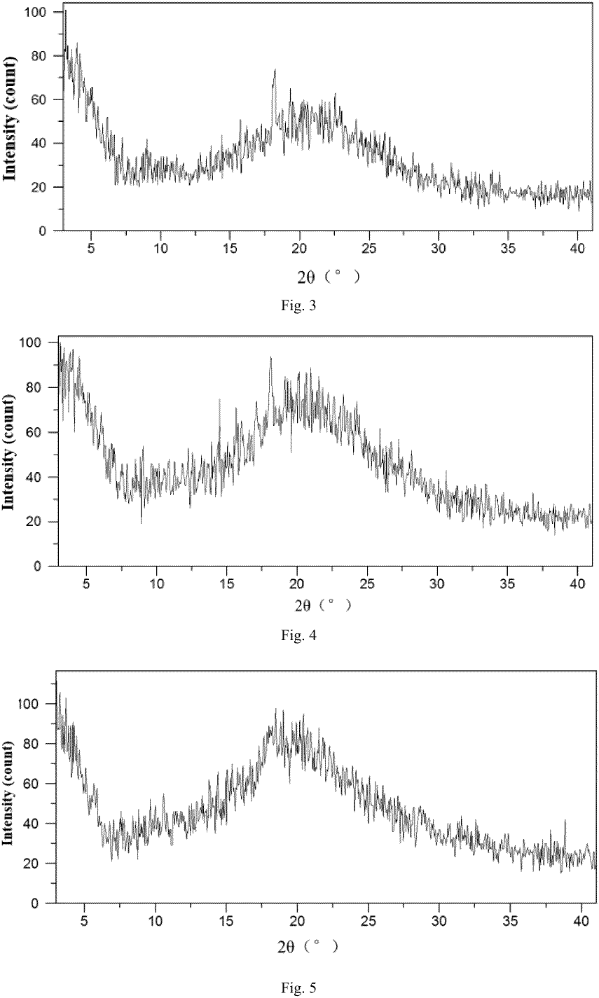

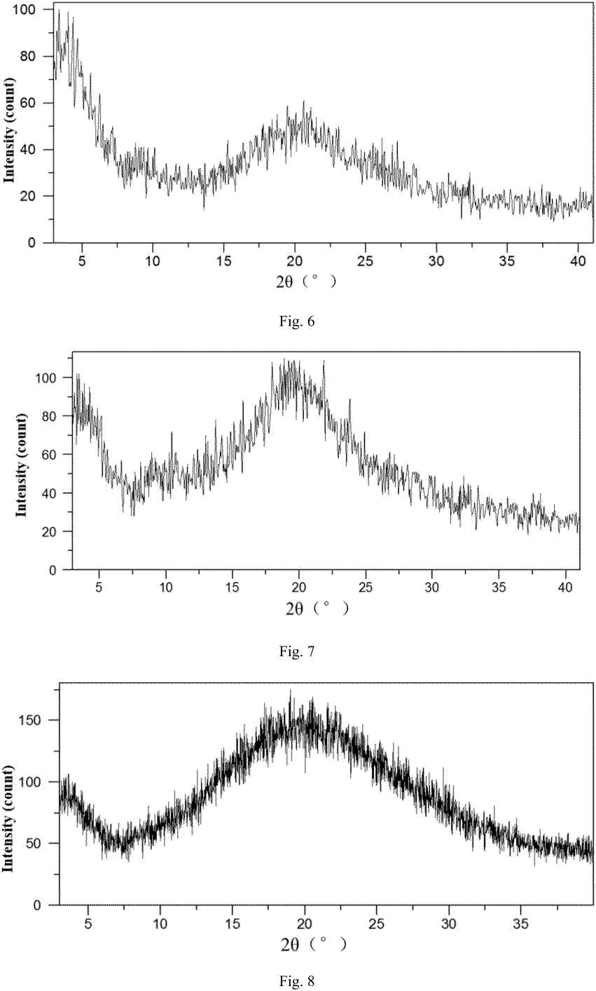

XRPD patterns of the sulfate, the phosphate, the isethionate, the α-naphthalenesulfonate, and the L-malate are respectively shown in FIG. 3 to FIG. 7.

### Example 3 Preparation of a Monohydrochloride of the Compound Represented by Formula 1

The compound represented by formula 1 (2 g) and toluene (10 mL) were placed in a 20 mL vial and shaken at the room temperature until the solid was fully dissolved. The clear solution was placed in a 100 mL double-layer glass jacketed reactor, a 4 mol/L hydrogen chloride-ethyl acetate solution (0.99 mL) was placed in the reactor, and the reaction solution was stirred for reaction for 15 min. N-heptane (40 mL) was placed in the reactor, and the reaction solution was stirred for curing at the room temperature for 2 h. After being cured, the reaction solution was subjected to suction filtration, and an obtained wet filter cake was dried in vacuum at 40° C. for 19 h to yield a white solid powdery monohydrochloride of the compound represented by formula 1 (1.97 g).

¹HNMR (400 MHz, DMSO-d₆): 10.51 (s, 1H), 9.06 (s, 1H), 8.54 (s, 1H), 8.21 (s, 1H), 7.13-7.52 (m, 4H), 4.51-4.94 (m, 1H), 3.88-4.19 (m, 1H), 3.50-3.81 (m, 3H), 2.97-3.40 (m, 4H), 2.73-2.83 (m, 1H), 2.23-2.31 (m, 1H), 1.07-1.30 (m, 8H), 0.83-0.98 (m, 4H), 0.05 (q, J=5.2 Hz, 1H).

The XRPD pattern of the monohydrochloride of the compound represented by formula 1 is shown in FIG. 8.

### Example 4 Preparation of a Dihydrochloride of the Compound Represented by Formula 1

The compound represented by formula 1 (2 g) and toluene (10 mL) were placed in a 100 mL double-layer glass jacketed reactor and stirred at the room temperature until the solid was fully dissolved. A 4 mol/L hydrogen chloride-ethyl acetate solution (2.18 mL) was placed in the reactor, and the reaction solution was stirred for reaction for 15 min. N-heptane (40 mL) was placed in the reactor, and the reaction solution was stirred for curing at the room temperature for 4 h. After being cured, the reaction solution was subjected to suction filtration, and an obtained wet filter cake was dried in vacuum at 40° C. for 6 h to yield a white solid powdery dihydrochloride of the compound represented by formula 1 (2.25 g).

¹HNMR (400 MHz, DMSO-d₆): 10.77 (s, 1H), 9.47 (s, 1H), 8.80 (s, 1H), 8.34 (s, 1H), 7.12-7.51 (m, 4H), 6.68 (s, 1H), 4.64-5.11 (m, 1H), 3.92-4.24 (m, 1H), 3.50-3.82 (m, 3H), 3.22-3.37 (m, 3H), 2.78-3.05 (m, 2H), 2.26-2.34 (m, 1H), 1.09-1.31 (m, 8H), 0.83-0.96 (m, 4H), 0.15 (q, J=5.2 Hz, 1H).

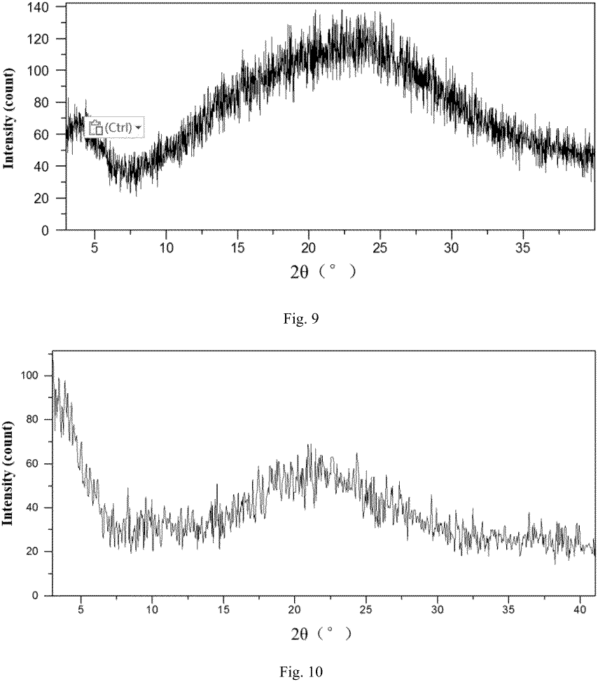

The XRPD pattern of the dihydrochloride of the compound represented by formula 1 is shown in FIG. 9.

### Example 5 Preparation of a Fumarate of the Compound Represented by Formula 1

The compound represented by formula 1 (25 mg) and isopropanol (1 mL) were placed in a 3 mL vial and magnetically stirred at the room temperature until the solid was fully dissolved. Solid fumaric acid (6.31 mg) was placed in the 3 mL vial, and the reaction solution was magnetically stirred for reaction. After the reaction solution was stirred for 18 h, n-heptane (2 mL) was placed in the 3 mL vial, and the reaction solution was stirred for 18 h. The reaction solution was subjected to suction filtration, and an obtained wet filter cake was dried in vacuum at 40° C. for 3 h to yield a white solid powdery fumarate of the compound represented by formula 1.

¹HNMR (400 MHz, DMSO-d₆): 10.49 (s, 1H), 8.20 (s, 1H), 7.34-7.48 (m, 4H), 6.52 (s, 2H), 4.37-4.76 (m, 1H), 3.88-4.18 (m, 1H), 3.70-3.81 (m, 2H), 3.34-3.54 (m, 2H), 3.03-3.21 (m, 4H), 2.90 (dd, J=11.6, 4.8 Hz, 1H), 2.76 (dd, J=16.4, 6.0 Hz, 1H), 2.22-2.30 (m, 1H), 1.04-1.32 (m, 8H), 0.85-0.93 (m, 4H), 0.08 (q, J=5.2 Hz, 1H).

The XRPD pattern of the fumarate of the compound represented by formula 1 is shown in FIG. 10.

In the present application, as demonstrated by Test Example 1 above, the compound represented by formula 1 of the present application has an inhibiting effect on the AKT kinase activity, and correspondingly, the pharmaceutically acceptable salt, such as a fumarate, a mesylate, an isethionate, an α-naphthalenesulfonate, a p-toluenesulfonate, a 1,2-ethanedisulphonate, an oxalate, a maleate, a citrate, a succinate, an L-(+)-tartrate, a hippurate, an L-ascorbate, an L-malate, a benzoate, a gentisate, a monohydrochloride, a dihydrochloride, a sulfate, and a phosphate, of the compound represented by formula 1 of the present application also has an inhibiting effect on the AKT kinase activity. Therefore, the pharmaceutically acceptable salt of the compound represented by formula 1 and the pharmaceutical composition comprising the salt of the present application can be used for preventing and/or treating an AKT protein kinase-mediated disease or disease state, and further can be used for preparing a medicament for preventing and/or treating an AKT protein kinase-mediated disease or disease state. Much further, compared with the compound represented by formula 1, the pharmaceutically acceptable salt of the compound represented by formula 1 of the present application has higher stability and better physical and chemical properties than the compound represented by formula 1, so it is more favorable for production and application.

The above are preferred embodiments of the present application only, but are not intended to limit the present application. Any modification, equivalent replacement, and improvement made within the spirit and principle of the present application shall fall within the protection scope of the present application.

What is claimed is:

1. A pharmaceutically acceptable salt of a compound represented by formula 1, the pharmaceutically acceptable salt selected from a salt of organic acid or a salt of inorganic acid, wherein the salt of organic acid is selected from the group consisting of a fumarate, a mesylate, an isethionate, an α-naphthalenesulfonate, a p-toluenesulfonate, a 1,2-ethanedisulphonate, an oxalate, a maleate, a citrate, a succinate, an L-(+)-tartrate, a hippurate, an L-ascorbate, an L-malate, a benzoate, and a gentisate, and the salt of inorganic acid is selected from the group consisting of a hydrochloride, a sulfate, and a phosphate, 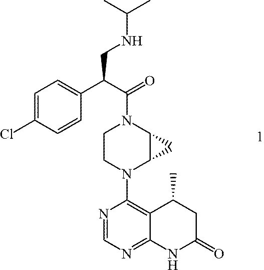 [SMILES: CC(C)NC[C@@H](C(=O)N1CCN(c2ncnc3c2[C@H](C)CC(=O)N3)[C@H]2C[C@H]21)c1ccc(Cl)cc1]
.

2. The pharmaceutically acceptable salt according to claim 1, wherein the pharmaceutically acceptable salt is selected from the group consisting of a fumarate and a hydrochloride.

3. The pharmaceutically acceptable salt according to claim 1, wherein a ratio of the compound represented by formula 1 to the organic acid is 1: 1.

4. The pharmaceutically acceptable salt according to claim 1, wherein a ratio of the compound represented by formula 1 to hydrogen chloride is 1: 1 or 1: 2.

5. The pharmaceutically acceptable salt according to claim 1, wherein a ratio of the compound represented by formula 1 to hydrogen chloride is 1: 2.

6. The pharmaceutically acceptable salt according to claim 1, wherein the pharmaceutically acceptable salt is a fumarate, and a molar ratio of the compound represented by formula 1 to fumaric acid is 1: 1.

7. A preparation method of the pharmaceutically acceptable salt of the compound represented by formula 1 according to claim 1, comprising a step of a salification reation of the compound represented by formula 1 with a corresponding acid.

8. The preparation method according to claim 7, wherein a solvent for the salification reaction is selected from the group consisting of a mixed solvent of an alcohol solvent and an alkane solvent, a mixed solvent of a ketone solvent and an alkane solvent, a mixed solvent of an ester solvent and an alkane solvent, a mixed solvent of a benzene solvent and an alkane solvent, and a mixed solvent of a halogenated hydrocarbon solvent and an alkane solvent.

9. A pharmaceutical composition comprising the pharmaceutically acceptable salt according to claim 1.

10. The pharmaceutically acceptable salt according to claim 1 or a pharmaceutical composition thereof for a use as a medicament, wherein the pharmaceutical composition comprises the pharmaceutically acceptable salt.

11. A method of a use of the pharmaceutically acceptable salt according to claim 1 or a pharmaceutical composition thereof in a prevention and/or a treatment of an AKT protein kinase-mediated disease or disease state, wherein the pharmaceutical composition comprises the pharmaceutically acceptable salt.

12. A method of a use of the pharmaceutically acceptable salt according to claim 1 or a pharmaceutical composition thereof in a preparation of a medicament for preventing and/or treating an AKT protein kinase-mediated disease or disease state, wherein the pharmaceutical composition comprises the pharmaceutically acceptable salt.

13. The method of the use according to claim 11, wherein the AKT protein kinase-mediated disease or disease state is a cancer, preferably a breast cancer, a prostate cancer, or an ovarian cancer, and more preferably the prostate cancer.

14. A method for preventing and/or treating an AKT protein kinase-mediated disease or disease state, comprising a step of administering the pharmaceutically acceptable salt according to claim 1 or a pharmaceutical composition thereof to a subject in need, wherein the pharmaceutical composition comprises the pharmaceutically acceptable salt.

15. The method according to claim 14, wherein the AKT protein kinase-mediated disease or disease state is a cancer, preferably a breast cancer, a prostate cancer, or an ovarian cancer, and more preferably the prostate cancer.

16. The method of the use according to claim 12, wherein the AKT protein kinase-mediated disease or disease state is a cancer, preferably a breast cancer, a prostate cancer, or an ovarian cancer, and more preferably the prostate cancer.
